(19) United States
(12) Patent Application Publication
(10) Pub. No.: US 2023/0242520 A1
(43) Pub. Date: Aug. 3, 2023

(54) MALONITRILE DERIVATIVES

(71) Applicant: Hoffmann-La Roche Inc., Little Falls, NJ (US)

(72) Inventors: Guillaume DÉCORET, Eschentzwiller (FR); Guido GALLEY, Rheinfelden (DE); Katrin GROEBKE ZBINDEN, Liestal (CH); Wolfgang GUBA, Müllheim (DE); Daniel HUNZIKER, Möhlin (CH)

(73) Assignee: Hoffmann-La Roche Inc., Little Falls, NJ (US)

(21) Appl. No.: 17/999,196

(22) PCT Filed: May 18, 2021
(86) PCT No.: PCT/EP2021/063037

§ 371 Date: Nov. 17, 2022

(30) Foreign Application Priority Data
May 20, 2020 (EP) 20175655.8

(51) Int. Cl. C07D 413/12; C07D 233/64; C07D 261/08; C07D 249/04; C07D 263/32; C07D 275/02; C07D 495/04

(52) U.S. Cl. CPC C07D 413/12; C07D 233/64; C07D 261/08; C07D 249/04; C07D 263/32; C07D 275/02; C07D 495/04

(57) ABSTRACT

The invention relates to a compound of formula (I) wherein R¹-R² are as defined in the description and in the claims The compound of formula (I) can be used as a medicament.

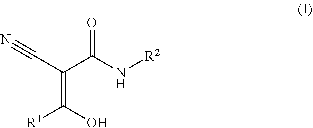

The present invention relates to organic compounds useful for therapy and/or prophylaxis in a mammal, and in particular to compounds that modulate cGAS activity.

The invention relates in particular to a compound of formula (I)

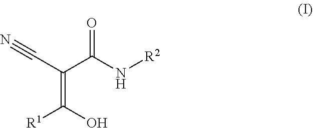

wherein
R¹ is alkylisoxazolyl, alkyltriazolyl, alkylimidazolyl, alkylisothiazolyl or oxazolyl; and
R² is alkyl-dioxido-7,8-dihydro-5H-thiopyrano[4,3-d]pyrimidinyl, alkoxy-5,6,7,8-tetrahydroquinazolinyl, phenyl-5,6,7,8-tetrahydroquinazolinyl, (alkoxyphenyl)-1-oxo-1,2-dihydroisoquinolinyl, dialkylphenylaminocarbonylpyridyl, alkoxypyrazinyl, pyridyl, naphtalenyl, alkoxypyridyl, alkoxypyridazinyl, oxo-pyridyl, (N-alkyl)oxo-pyridyl, oxo-indanyl, haloalkylphenyl, alkoxycarbonylphenyl, (alkoxycarbonyl)alkylthiophenyl or (alkoxycarbonyl)thiophenyl;
or a pharmaceutically acceptable salt, stereoisomer or tautomer thereof.

Cytokines are responsible for modulation of the innate immune response and the dysregulation of pro-inflammatory cytokines has been associated with severe systemic inflammation and autoimmune diseases, many of which lack efficient therapy as of today.

Vertebrates possess an innate and adaptive immune system as protection against pathogens and other challenges. The innate immune system is an evolutionary old system that is present beyond vertebrates. Unlike the adaptive immune system, it does not require priming or training, but works as a general physical barrier (e.g. skin) or by detection of specific patterns. One universal pattern to trigger the innate immune system is the detection of cytosolic double stranded DNA, which leads to Type I Interferon response. Sources of cytosolic dsDNA could be from bacterial or viral infection but as well accumulated self-DNA.

The cytosolic enzyme cyclic GMP-AMP Synthase (cGAS) is a sensor for cytosolic double stranded DNA. Binding of dsDNA results in the generation of the cyclic di-nucleotide 2,3-cGAMP by enzymatic linkage of ATP and GTP. 2,3-cGAMP acts as secondary messenger and binds to the Stimulator of Interferon Genes (STING), which resides in the endoplasmatic reticulum. Upon binding of 2,3-cGAMP, STING translocates to the perinuclear Golgi, where it associates with the TANK binding kinase 1 (TBK1) and recruits and phosphorylates Interferon Response Factor 3 (IRF3). Ultimately this results in the production Type I Interferon (I IFN), other cytokines like IL-6, TNFα, IL1β and chemokines—essential factors for host defense against invading pathogens. However, inappropriate or chronic production of type I IFN and other pro-inflammatory cytokines are associated with severe systemic inflammation and autoimmune diseases. For instance, IFN signaling is involved in SLE, cutaneous skin diseases (dermatomyositis, and cutaneous lupus), interstitial pulmonary fibrosis, Sjogren syndrome, and type I diabetes (G. Trinchieri, J Exp Med. 2010 207(10): 2053-63). Other pro-inflammatory cytokine such as TNFα and IL1β play an important role in inflammatory bowel disease, NASH, juvenile inflammatory arthritis, ankylosing spondylitis and gout.

Chronic activation of cGAS/STING causes severe systemic inflammation. Evidence for its role in inflammation in the clinic comes from monogenic diseases. Patients with deficiencies in nucleic acid modifying enzymes, like Trex1, RNaseH2 and SAMHD1, suffer from Aicardi-Goutieres syndrome (AGS). The involvement of cGAS/STING was supported in Trex1 deficient mice that serve as a model for AGS.

Inhibition of the cGAS pathway which is upstream from the disease mediating cytokines is therefore a novel strategy in treating patients from multiple autoimmune diseases. Indications could include those linked to IFN signaling or those driven by TNFα and IL1β.

As of today many diseases caused by dysregulation of the innate immune system lack efficient therapies.

The compound of the invention binds to cGAS and modulates its activity.

The compound of formula (I) is particularly useful in the treatment or prophylaxis of e.g. systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl, isopropyl, butyl, isobutyl, tert.-butyl and pentyl. Methyl is a particular example of “alkyl” in the compound of formula (I).

The term “alkoxy” or “alkyloxy”, alone or in combination, signifies a group of the formula alkyl—O—in which the term “alkyl” has the previously given significance, such as methoxy, ethoxy, n-propoxy, isopropoxy, n-butoxy, isobutoxy, sec-butoxy and tert.-butoxy. A particular example of “alkoxy” is methoxy.

The term “oxy”, alone or in combination, signifies the —O— group.

The term “oxo”, alone or in combination, signifies the ═O group.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. A particular “haloalkyl” is fluoromethyl.

The terms “hydroxyl” and “hydroxy”, alone or in combination, signify the —OH group.

The term “carbonyl”, alone or in combination, signifies the —C(O)— group.

The term “amino”, alone or in combination, signifies the primary amino group (—NH₂), the secondary amino group (—NH—), or the tertiary amino group (—N—).

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from the addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of trifluoroacetic acid, hydrochloric acid, hydrobromic acid, sulfuric acid, phosphoric acid and methanesulfonic acid.

The compound of formula (I) can exist as a tautomer (I′), i.e. a structural isomer which interconverts with the the compound of formula (I), in particular in solution.

The tautomeric equilibrium of the compound of formula (I) with its tautomeric form (I′) can be represented as follows:

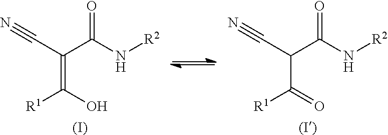

The compound of formula (I) can exist as a stereoisomer (I″), i.e. a structural isomer which interconverts with the the compound of formula (I), in particular in solution.

The isomeric equilibrium of the compound of formula (I) with its stereoisomeric form (I″) can be represented as follows:

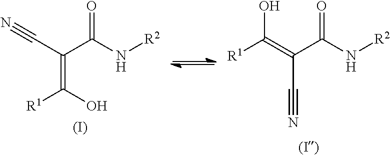

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, N.Y.) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

The invention thus relates to:

A compound according to the invention, wherein R¹ is methylisoxazolyl, methyltriazolyl, methylimidazolyl, methylisothiazolyl or oxazolyl;

A compound according to the invention wherein R¹ is alkylisoxazolyl;

A compound according to the invention wherein R¹ is methylisoxazolyl;

A compound according to the invention wherein R² is methyl-dioxido-7,8-dihydro-5H-thiopyrano[4,3-d]pyrimidinyl, methoxy-5,6,7,8-tetrahydroquinazolinyl, phenyl-5,6,7,8-tetrahydroquinazolinyl, (methoxyphenyl)-1-oxo-1,2-dihydroisoquinolinyl, dimethylphenylaminocarbonylpyridyl, methoxypyrazinyl, pyridyl, naphtalenyl, methoxypyridyl, methoxypyridazinyl, oxo-pyridyl, (N-methyl)oxo-pyridyl, oxo-indanyl, trifluoromethylphenyl, methyloxycarbonylphenyl, (ethoxycarbonyl)methylthiophenyl or (methoxycarbonyl)thiophenyl;

A compound according to the invention wherein R² is oxo-pyridyl, (N-alkyl)oxo-pyridyl or oxoindanyl;

A compound according to the invention wherein R² is oxo-pyridyl, (N-methyl)oxo-pyridyl or oxoindanyl.

The invention further relates to a compound of formula (I) selected from
(Z)-2-cyano-3-hydroxy-N-(4-methyl-6,6-dioxido-7,8-dihydro-5H-thiopyrano[4,3-d]pyrimidin-2-yl)-3-(5-methylisoxazol-4-yl)acrylamide;
(Z)-2-cyano-3-hydroxy-N-(4-methoxy-5,6,7,8-tetrahydroquinazolin-2-yl)-3-(5-methylisoxazol-4-yl)acrylamide;
(Z)-2-cyano-3-hydroxy-3-(5-methylisoxazol-4-yl)-N-(4-phenyl-5,6,7,8-tetrahydroquinazolin-2-yl)acrylamide;
(Z)-2-cyano-3-hydroxy-N-(3-(3-methoxyphenyl)-1-oxo-1,2-dihydroisoquinolin-6-yl)-3-(5-methylisoxazol-4-yl)acrylamide;
(Z)-5-(2-cyano-3-hydroxy-3-(5-methylisoxazol-4-yl)acrylamido)-N-(2,6-dimethylphenyl)picolinamide;
(Z)-2-cyano-3-hydroxy-N-(5-methoxypyrazin-2-yl)-3-(5-methylisoxazol-4-yl)prop-2-enamide;
(Z)-2-cyano-3-hydroxy-3-(5-methylisoxazol-4-yl)-N-(4-pyridyl)prop-2-enamide;
(Z)-2-cyano-3-hydroxy-3-(5-methylisoxazol-4-yl)-N-(naphthalen-2-yl)acrylamide;
(Z)-2-cyano-3-hydroxy-3-(5-methylisoxazol-4-yl)-N-(naphthalen-1 -yl)acrylamide;
(Z)-2-cyano-3-hydroxy-N-(6-methoxy-3-pyridyl)-3-(5-methylisoxazol-4-yl)prop-2-enamide;
(Z)-2-cyano-3-hydroxy-N-(6-methoxypyridazin-3-yl)-3-(5-methylisoxazol-4-yl)prop-2-enamide;
(Z)-2-cyano-3-hydroxy-3-(5-methylisoxazol-4-yl)-N-(2-oxo-1H-pyridin-4-yl)prop-2-enamide;
(Z)-2-cyano-3-hydroxy-3-(5-methylisoxazol-4-yl)-N-(1-methyl-2-oxo-4-pyridyl)prop-2-enamide;
(Z)-2-cyano-3-hydroxy-3-(1-methyl-1H-1,2,3-triazol-5-yl)-N-(4-(trifluoromethyl)phenyl)acrylamide;
(Z)-2-cyano-3-hydroxy-3-(5-methylisoxazol-4-yl)-N-(1-oxoindan-5-yl)prop-2-enamide;
(Z)-2-cyano-3-hydroxy-3-(1-methyl-1H-imidazol-5-yl)-N-(4-(trifluoromethyl)phenyl)acrylamide;
(Z)-2-cyano-3-hydroxy-3-(5-methylisoxazol-4-yl)-N-(3-pyridyl)prop-2-enamide;
(Z)-2-cyano-3-hydroxy-3-(5-methylisothiazol-4-yl)-N-(4-(trifluoromethyl)phenyl)acrylamide;
(Z)-2-cyano-3-hydroxy-3-(4-methylisoxazol-5-yl)-N-(4-(trifluoromethyl)phenyl)acrylamide;
(Z)-2-cyano-3-hydroxy-3-(oxazol-5-yl)-N-(4-(trifluoromethyl)phenyl)acrylamide;
methyl (Z)-4-(2-cyano-3-hydroxy-3-(3-methylisoxazol-4-yl)acryl amido)benzoate;
(Z)-2-cyano-3-hydroxy-3-(3-methylisoxazol-4-yl)-N-(4-(trifluoromethyl)phenyl)acrylamide;
ethyl (Z)-2-(2-cyano-3-hydroxy-3-(5-methylisoxazol-4-yl)acrylamido)-4-methylthiophene-3-carboxylate; and
methyl (Z)-3-(2-cyano-3-hydroxy-3-(5-methylisoxazol-4-yl)acrylamido)thiophene-2-carboxylate;
or a pharmaceutically acceptable salt, stereosiomer or tautomer thereof.

The invention further relates to a compound of formula (I) selected from
(Z)-2-cyano-3-hydroxy-3-(5-methylisoxazol-4-yl)-N-(2-oxo-1H-pyridin-4-yl)prop-2-enamide;
(Z)-2-cyano-3-hydroxy-3-(5-methylisoxazol-4-yl)-N-(1-methyl-2-oxo-4-pyridyl)prop-2-enamide; and
(Z)-2-cyano-3-hydroxy-3-(5-methylisoxazol-4-yl)-N-(1-oxoindan-5-yl)prop-2-enamide;
or a pharmaceutically acceptable salt, stereoisomer or tautomer thereof.

The synthesis of the compound of formula (I) can, for example, be accomplished according to scheme 1.

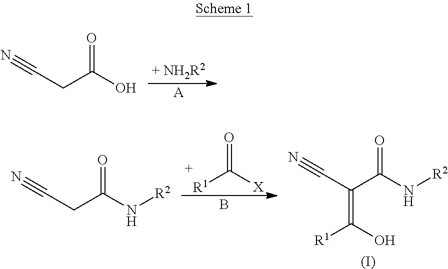

In scheme 1, R¹ and R² are as defined above; X is a leaving group, such as halogen, mesylate or tosylate.

Step A: A suitable amine is coupled with cyano acetic acid, using a coupling reagent such as DCC, HATU, EDCI or propyl phosphonic anhydride optionally in the presence of a base such as triethylamine, DIPEA or pyridine in a solvent such as dichloromethane, THF, acetonitrile, ethyl acetate or DMF, or alternatively by activating the acid via the acid chloride with oxalyl chloride/DMF, thionyl chloride/DMF or methanesulfonyl chloride/3-methylpyridine optionally in the presence of a base such as triethylamine, DIPEA in a solvent such as acetonitrile, THF, dichloromethane, toluene or dioxane at a temperature between room temperature and around 80° C. for 2-12 hrs.

Step B: Compounds of formula (I) can be obtained by deprotonation of cyano acetamide with a base and subsequent reaction with an acid substituted with a leaving group X, such as a halogen, mesylate or tosylate in a solvent such as THF, dichloromethane or a mixture thereof. Convenient conditions are the use of NaH as a base in a mixture of THF and dichloromethane at around room temperature for between around 5-20 hrs. Conveniently X is a halogen, in particular chloride.

The invention thus also relates to a process for the preparation of a compound according to the invention, comprising the coupling of a compound of formula (A1)

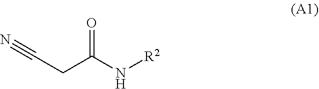

with a compound of formula (A2)

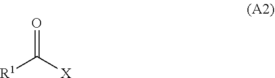

in the presence of a base;
wherein R¹ and R² are as defined above and X is a leaving group such as a halogen, mesylate or tosylate.

Conveniently X is a halogen, in particular chloride.

The coupling can conveniently be carried out in a solvent. The solvent can be for example THF, dichloromethane or a mixture thereof.

In the coupling the base can be for example NaH or tert-butoxide. Conveniently the base is NaH.

Convenient conditions for the coupling can be between around 0° C.-100° C., particularly between around 5° C.-80° C., more particularly between around 10° C.-50° C.

Preferred conditions for the coupling are the use of NaH in a mixture of THF and dichloromethane at around room temperature for between around 1-24 hrs, in particular between around 5-20 hrs.

The invention also relates to a compound according to the invention when manufactured according to a process of the invention.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal, epidural and intranasal, and if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The invention also relates in particular to:

A compound of formula (I) for use as therapeutically active substance;

A pharmaceutical composition comprising a compound of formula (I) and a therapeutically inert carrier;

A compound of formula (I) for use in the treatment of a disease modulated by cGAS;

The use of a compound of formula (I) for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);

The use of a compound of formula (I) for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS);

A compound of formula (I) for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS); and

A method for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS), which method comprises administering an effective amount of a compound of formula (I) to a patient in need thereof.

The invention will now be illustrated by the following examples which have no limiting character.

### EXAMPLES

### Abbreviations

ATP=adenosine triphosphate; BSA=bovine serum albumine; DCC=dicyclohexylurea;
DIPEA=diisopropylamine; DMF=dimethylformamide; DMSO=dimethyl sulfoxide;
DNA=deoxyribonucleic acid; EDCI=1-ethyl-3-(3-dimethylaminopropyl)carbodiimide;
ESI=electrospray ionization; EtOAc=ethyl acetate; GTP=guanosine triphosphate;
HATU=hexafluorophosphate azabenzotriazole tetramethyl uronium; MeOH=methanol;
MS=mass spectrometry; RT=room temperature; SD=standard deviation; THF=tetrahydrofuran; TRIS=tris(hydroxymethyl)aminomethane.

### Example 1

### (Z)-2-Cyano-3-hydroxy-3-(1-methyl-1H-imidazol-5-yl)-N-(4-(trifluoromethyl)phenyl)acrylamide

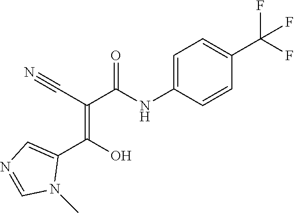 [SMILES: Cn1cncc1/C(O)=C(\C#N)C(=O)Nc1ccc(C(F)(F)F)cc1]

### Step 1: 1-Methyl-1H-imidazole-5-carbonyl chloride

To a stirred suspension of 1-methyl-1H-imidazole-5-carboxylic acid (500 mg, 3.96 mmol) at room temperature (RT) in THF (8 mL) under an argon atmosphere were added 2 drops of DMF followed by oxalyl chloride (554 mg, 369 μl, 4.36 mmol). The mixture immediately turned dark. Stirring at RT was continued for 3 h 30. The reaction mixture was concentrated to dryness to leave the crude product as a brown solid (568 mg).

### Step 2: 2-Cyano-N-(4-(trifluoromethyl)phenyl)acetamide

To a stirred solution of 2-cyanoacetic acid (4.17 g, 49 mmol) at RT in ethyl acetate (85 mL) under an argon atmosphere was added 4-(trifluoromethyl)aniline (5 g, 3.9 mL, 31 mmol) in one portion. To the resulting clear, light yellow solution was added dropwise a solution of DCC (7.05 g, 34.2 mmol) in ethylacetate (45 mL) for 20 min. When the addition was complete (a white solid soon precipitated out: urea from DCC), stirring at RT was continued overnight. The insoluble urea was filtered off and the cake (white powder) was washed with EtOAc. The filtrate was washed with saturated aq. NaHCO_{3,} then with 1 N HCl, and then with brine. The organic phase was dried (MgSO₄), filtered and concentrated to leave the crude product as a light yellow solid. The crude product was taken up in isopropanol (85 mL) and the suspension was heated to 80° C. (oil bath temperature) under stirring until a clear light yellow solution was obtained. The mixture was allowed to cool to RT, a solid precipitated out. The suspension was stirred at RT for another 1 h. The product was collected by filtration, washed with 2-propanol and dried to provide the title compound as a white solid. The filtrate still contains some product. It was concentrated to leave a light yellow sticky solid. This was triturated in 10 mL of MeOH. The suspension was stirred at RT for 1 h. The solid was collected by filtration, washed with MeOH and dried. Another 1.03 g of the title compound was isolated as a white solid. Total yield: 5.58 g, 77%. MS: 227.1 [M-H]-ESI neg.

### Step 3: (Z)-2-Cyano-3-hydroxy-3-(1-methyl-1H-imidazol-5-yl)-N-(4-(trifluoromethyl)-phenyl)acrylamide

To a stirred solution of 2-cyano-N-(4-(trifluoromethyl)phenyl)acetamide (200 mg, 877 μmol) at RT in THF (5 mL) under an argon atmosphere was added sodium hydride 60% dispersion in mineral oil (80.6 mg, 2.02 mmol). After stirring for 10 mins, a solution of 1-methyl-1H-imidazole-5-carbonyl chloride (158 mg, 877 μmol) in CH₂Cl₂ (0.5 ml) was added in one portion. Stirring at RT was continued for 17 hrs. The mixture was carefully treated with 0.5 M HCl (3 mL), diluted with CH₂Cl_{2.} A precipitate formed. The solid was collected by filtration and washed with CH₂Cl_{2.} This brown solid was then triturated in ˜5 mL of MeOH. The suspension was stirred at RT for 17 hrs. The product was collected by filtration, washed with MeOH and dried to provide the title compound (159 mg, 51%) as light brown solid. MS: 337.1 [M+H]+ ESI pos.

In analogy to the procedures described in example 1, examples 2-24 (Table 1) where prepared using a suitable acid in the first step and a suitable amine in the second step.

TABLE 1

| Ex. | Systematic name | Structure | MS |
|---|---|---|---|
| 2 | (Z)-2-cyano-3-hydroxy-3- (5-methylisoxazol-4-yl)- N-(3-pyridyl)prop-2- | 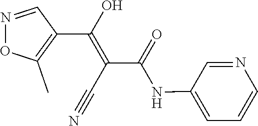 [SMILES: Cc1oncc1/C(O)=C(\C#N)C(=O)Nc1cccnc1] | 271.1 [M + H]+ |
| 3 | (Z)-2-cyano-3-hydroxy-3- (5-methylisoxazol-4-yl)- N-(1-oxoindan-5-yl)prop- 2-enamide | 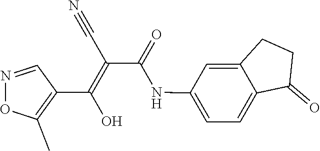 [SMILES: Cc1oncc1/C(O)=C(\C#N)C(=O)Nc1ccc2c(c1)CCC2=O] | 324.4 [M + H]+ |
| 4 | (Z)-2-cyano-3-hydroxy-3- (1-methyl-1H-1,2,3- triazol-5-yl)-N-(4- (trifluoromethyl)phenyl) acrylamide | 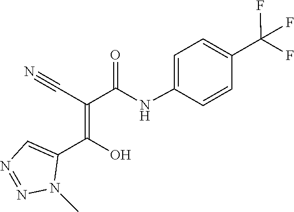 [SMILES: Cn1nncc1/C(O)=C(\C#N)C(=O)Nc1ccc(C(F)(F)F)cc1] | 338.1 [M + H]+ |
| 5 | (Z)-2-cyano-3-hydroxy-3- (5-methylisoxazol-4-yl)- N-(1-methyl-2-oxo-4- pyridyl)prop-2-enamide | 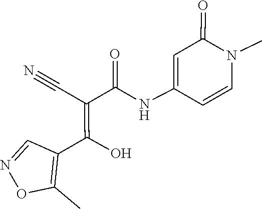 [SMILES: Cc1oncc1/C(O)=C(\C#N)C(=O)Nc1ccn(C)c(=O)c1] | 301.0 [M + H]+ |
| 6 | (Z)-2-cyano-3-hydroxy-3- (5-methylisoxazol-4-yl)- N-(2-oxo-1H-pyridin-4- yl)prop-2-enamide | 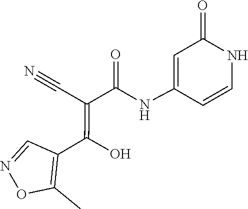 [SMILES: Cc1oncc1/C(O)=C(\C#N)C(=O)Nc1cc[nH]c(=O)c1] | 287.1 [M + H]+ |
| 7 | (Z)-2-cyano-3-hydroxy-N- (6-methoxypyridazin-3- yl)-3-(5-methylisoxazol-4- yl)prop-2-enamide | 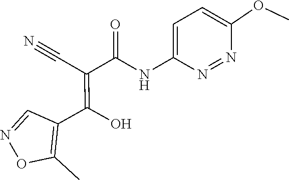 [SMILES: COc1ccc(NC(=O)/C(C#N)=C(\O)c2cnoc2C)nn1] | 302.1 [M + H]+ |
| 8 | (Z)-2-cyano- 3-hydroxy-N- (6-methoxy- 3-pyridyl)-3- (5-methylisoxazol-4- yl)prop-2-enamide | 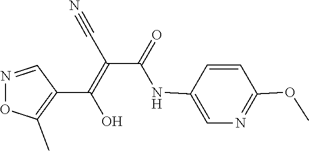 [SMILES: COc1ccc(NC(=O)/C(C#N)=C(\O)c2cnoc2C)cn1] | 301.4 [M + H]+ |
| 9 | methyl (Z)-3-(2-cyano-3- hydroxy-3-(5- methylisoxazol-4- yl)acrylamido) thiophene- 2-carboxylate | 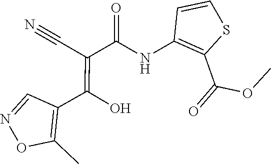 [SMILES: COC(=O)c1sccc1NC(=O)/C(C#N)=C(\O)c1cnoc1C] | 334.1 [M + H]+ |
| 10 | ethyl (Z)-2-(2-cyano-3- hydroxy-3-(5- methylisoxazol-4- yl)acrylamido)-4- methylthiophene-3- carboxylate | 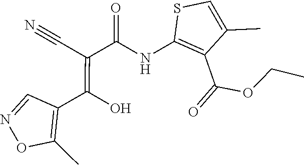 [SMILES: CCOC(=O)c1c(C)csc1NC(=O)/C(C#N)=C(\O)c1cnoc1C] | 362.1 [M + H]+ |
| 11 | (Z)-2-cyano-3-hydroxy-3- (3-methylisoxazol-4-yl)- N-(4- (trifluoromethyl)phenyl) acrylamide | 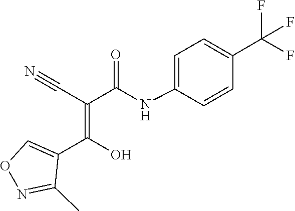 [SMILES: Cc1nocc1/C(O)=C(\C#N)C(=O)Nc1ccc(C(F)(F)F)cc1] | 338.1 [M + H]+ |
| 12 | methyl (Z)-4- (2-cyano-3- hydroxy-3-(3- methylisoxazol-4- yl)acrylamido) benzoate | 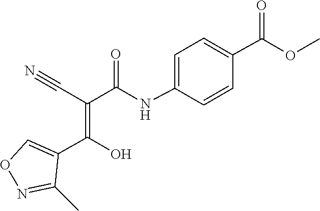 [SMILES: COC(=O)c1ccc(NC(=O)/C(C#N)=C(\O)c2conc2C)cc1] | 328.1 [M + H]+ |
| 13 | (Z)-2-cyano-3-hydroxy-3- (oxazol-5-yl)-N-(4- (trifluoromethyl)phenyl) acrylamide | 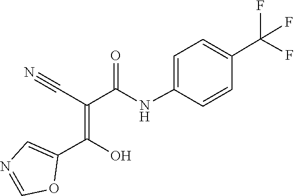 [SMILES: N#C/C(C(=O)Nc1ccc(C(F)(F)F)cc1)=C(/O)c1cnco1] | 324.1 [M + H]+ |
| 14 | (Z)-2-cyano-3-hydroxy-3- (4-methylisoxazol-5-yl)- N-(4- (trifluoromethyl)phenyl) acrylamide | 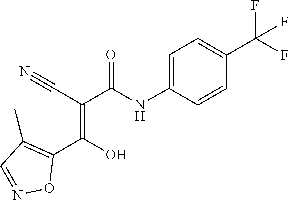 [SMILES: Cc1cnoc1/C(O)=C(\C#N)C(=O)Nc1ccc(C(F)(F)F)cc1] | 338.1 [M + H]+ |
| 15 | (Z)-2-cyano-3-hydroxy-3- (5-methylisothiazol-4-yl)- N-(4- (trifluoromethyl)phenyl) acrylamide | 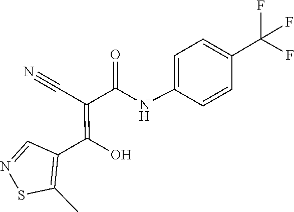 [SMILES: Cc1sncc1/C(O)=C(\C#N)C(=O)Nc1ccc(C(F)(F)F)cc1] | 354.1 [M + H]+ |
| 16 | (Z)-2-cyano-3-hydroxy-3- (5-methylisoxazol-4-yl)- N-(naphthalen-1- yl)acrylamide | 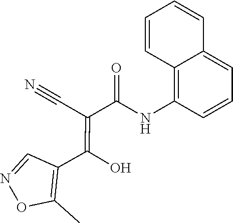 [SMILES: Cc1oncc1/C(O)=C(\C#N)C(=O)Nc1cccc2ccccc12] | 320.1 [M + H]+ |
| 17 | (Z)-2-cyano-3-hydroxy-3- (5-methylisoxazol-4-yl)- N-(naphthalen-2- yl)acrylamide | 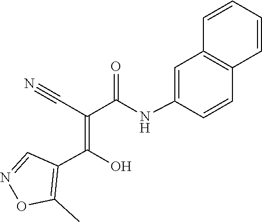 [SMILES: Cc1oncc1/C(O)=C(\C#N)C(=O)Nc1ccc2ccccc2c1] | 320.1 [M + H]+ |
| 18 | (Z)-2-cyano-3-hydroxy-3- (5-methylisoxazol-4-yl)- N-(4-pyridyl)prop-2- enamide | 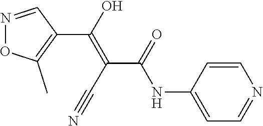 [SMILES: Cc1oncc1/C(O)=C(\C#N)C(=O)Nc1ccncc1] | 271.1 [M + H]+ |
| 19 | (Z)-2-cyano-3-hydroxy-N- (5-methoxypyrazin-2-yl)- 3-(5-methylisoxazol-4- yl)prop-2-enamide | 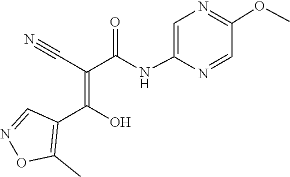 [SMILES: COc1cnc(NC(=O)/C(C#N)=C(\O)c2cnoc2C)cn1] | 302.1 [M + H]+ |
| 20 | (Z)-5-(2-cyano-3-hydroxy- 3-(5-methylisoxazol-4- yl)acrylamido)-N-(2,6- dimethylphenyl) picolinamide | 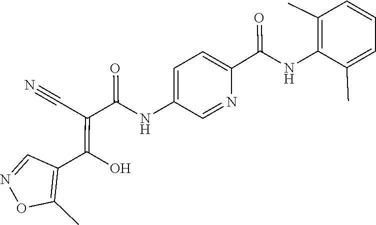 [SMILES: Cc1cccc(C)c1NC(=O)c1ccc(NC(=O)/C(C#N)=C(\O)c2cnoc2C)cn1] | 418.2 [M + H]+ |
| 21 | (Z)-2-cyano-3-hydroxy-N- (3-(3-methoxyphenyl)-1- oxo-1,2- dihydroisoquinolin-6-yl)- 3-(5-methylisoxazol-4- yl)acrylamide | 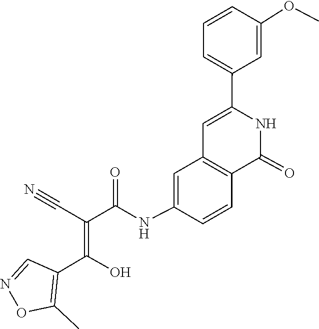 [SMILES: COc1cccc(-c2cc3cc(NC(=O)/C(C#N)=C(\O)c4cnoc4C)ccc3c(=O)[nH]2)c1] | 443.2 [M + H]+ |
| 22 | (Z)-2-cyano-3-hydroxy-3- (5-methylisoxazol-4-yl)- N-(4-phenyl-5,6,7,8- tetrahydroquinazolin-2- yl)acrylamide | 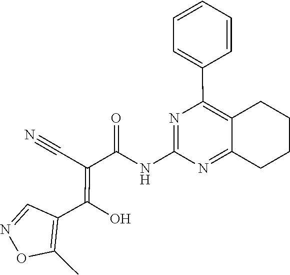 [SMILES: Cc1oncc1/C(O)=C(\C#N)C(=O)Nc1nc2c(c(-c3ccccc3)n1)CCCC2] | 402.2 [M + H]+ |
| 23 | (Z)-2-cyano-3-hydroxy-N- (4-methoxy-5,6,7,8- tetrahydroquinazolin-2- yl)-3-(5-methylisoxazol-4- yl)acrylamide | 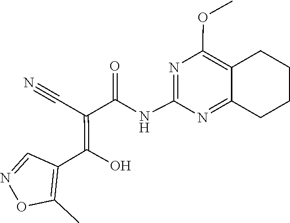 [SMILES: COc1nc(NC(=O)/C(C#N)=C(\O)c2cnoc2C)nc2c1CCCC2] | 356.2 [M + H]+ |
| 24 | (Z)-2-cyano-3-hydroxy-N- (4-methyl-6,6-dioxido-7,8- dihydro-5H- thiopyrano[4,3- d]pyrimidin-2-yl)-3-(5- methylisoxazol-4- yl)acrylamide | 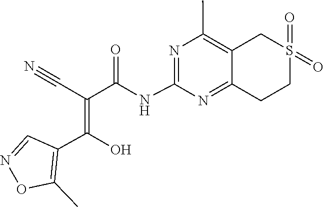 [SMILES: Cc1nc(NC(=O)/C(C#N)=C(\O)c2cnoc2C)nc2c1CS(=O)(=O)CC2] | 390.3 [M + H]+ |

### Example 25

### cGAS Activity Assay—Malachite Green

Compounds were tested for cGAS inhibition in a coupled enzymatic assay based on Phosphate detection by Malachite Green. Final assay conditions were 20 mM TRIS pH 7.5 (Applichem), 5 mM MgCl₂ (Sigma) and 0.01% BSA (Sigma) supplemented with 80 μM ATP (Sigma), 80 μM GTP (Sigma) and 100 nM Interferon Stimulating DNA (ISD) (Microsynth). Recombinantly expressed purified human cGAS (residues 161-522) was used at 25 nM.

All compounds were prepared as 10 mM stock solutions in DMSO and a 16 pt dilution series in DMSO with a dilution factor of 2.5 was prepared. 1 μL of DMSO dilution series was transferred to 32.3 μL reaction buffer, mixed by pipetting up/down, spun for 1 minute at 3000 rpm and was visually inspected for precipitation. 5 μL of 3-fold enzyme stock solution were transferred to an empty 384-well Black/Clear Flat Bottom Polystyrene NBS (Corning) rows 3-24. Rows 1-2 were filled with assay buffer. Plates were spun 10 seconds at 1000 rpm (164×g). 5 μL of compound intermediate dilution was added and mixed by pipetting up/down to rows 3-24. Rows 1-2 were filled with 3.1% DMSO assay buffer. Plates were spun 10 seconds at 1000 rpm (164×g). 5 μL 3-fold Nucleotide/DNA mix was added to all wells to start the reaction. Plates were spun 10 seconds at 1000 rpm (164×g) and incubated for 4 hour at room temperature (RT) in the dark. 5 μL 4 U/mL PPase (Sigma) were added to all wells. Plates spun 10 seconds at 1000 rpm (164×g). 10 μL BioMol green Solution (Enzo Life Sciences) was added to all wells. Plates spun 10 seconds at 1000 rpm (164×g) and incubated 30 minutes at RT in the dark. Absorbance data was collected 620 nm on an EnVision Multilable Reader (Perkin Elmer) and the following measurement settings were used: excitation filter photometric was 620 nm; excitation from the top; measurement height was 1 mm; number of flashes was 30; number of flashes integrated was 1.

All plates are checked for abnormalities and outliers in the Blank Control (no protein, row 1) and the Neutral Control (no compound, row 2) are excluded using the 3*SD rule. Data was normalized to 0 and 100% by Blank and Neutral Control and each curve was fitted and judged using the 4 parameter logistic equation to determine the IC50 for cGAS inhibition.

The results of this assay are provided in Table 2. Table 2 provides IC50 values (μM ) for cGAS inhibition obtained for particular examples of the present invention as measured by the above-described assay.

TABLE 2

| Example | IC50 cGAS (μM) |
|---|---|
| 1 | 1.85 |
| 2 | 1.57 |
| 3 | 0.22 |
| 4 | 6.31 |
| 5 | 0.17 |
| 6 | 0.18 |
| 7 | 1.02 |
| 8 | 1.34 |
| 9 | 1.27 |
| 10 | 0.82 |
| 11 | 3.99 |
| 12 | 1.13 |
| 13 | 7.19 |
| 14 | 8.48 |
| 15 | 4.76 |
| 16 | 1.79 |
| 17 | 1.11 |
| 18 | 0.62 |
| 19 | 0.84 |
| 20 | 0.50 |
| 21 | 1.06 |
| 22 | 0.97 |
| 23 | 5.21 |
| 24 | 0.45 |

### Example A

Film coated tablets containing the following ingredients can be manufactured in a conventional manner:

| Ingredients | Per tablet | |
|---|---|---|
| Kernel: | | |
| Compound of formula (1) | 10.0 mg | 200.0 mg |
| Microcrystalline cellulose | 23.5 mg | 43.5 mg |
| Lactose hydrous | 60.0 mg | 70.0 mg |
| Povidone K30 | 12.5 mg | 15.0 mg |
| Sodium starch glycolate | 12.5 mg | 17.0 mg |
| Magnesium stearate | 1.5 mg | 4.5 mg |
| (Kernel Weight) | 120.0 mg | 350.0 mg |
| Film Coat: | | |
| Hydroxypropyl methyl cellulose | 3.5 mg | 7.0 mg |
| Polyethylene glycol 6000 | 0.8 mg | 1.6 mg |
| Talc | 1.3 mg | 2.6 mg |
| Iron oxide (yellow) | 0.8 mg | 1.6 mg |
| Titan dioxide | 0.8 mg | 1.6 mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels of 120 or 350 mg respectively. The kernels are lacquered with an aq. solution/suspension of the above mentioned film coat.

### Example B

Capsules containing the following ingredients can be manufactured in a conventional manner:

| Ingredients | Per capsule |
|---|---|
| Compound of formula (I) | 25.0 mg |
| Lactose | 150.0 mg |
| Maize starch | 20.0 mg |
| Talc | 5.0 mg |

The components are sieved and mixed and filled into capsules of size 2.

### Example C

Injection solutions can have the following composition:

| | |
|---|---|
| Compound of formula (1) | 3.0 mg |
| Polyethylene glycol 400 | 150.0 mg |
| Acetic acid | q.s. ad pH 5.0 |
| Water for injection solutions | ad 1.0 ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

1. A compound of formula (I) 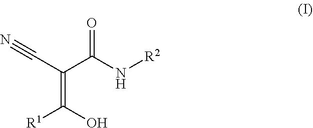
wherein
R¹ is alkylisoxazolyl, alkyltriazolyl, alkylimidazolyl, alkylisothiazolyl or oxazolyl; and
R² is alkyl-dioxido-7,8-dihydro-5H-thiopyrano[4,3-d]pyrimidinyl, alkoxy-5,6,7,8-tetrahydroquinazolinyl, phenyl-5,6,7,8-tetrahydroquinazolinyl, (alkoxyphenyl)-1-oxo-1,2-dihydroisoquinolinyl, dialkylphenylaminocarbonylpyridyl, alkoxypyrazinyl, pyridyl, naphtalenyl, alkoxypyridyl, alkoxypyridazinyl, oxo-pyridyl, (N-alkyl)oxo-pyridyl, oxo-indanyl, haloalkylphenyl, alkoxycarbonylphenyl, (alkoxycarbonyl)alkylthiophenyl or (alkoxycarbonyl)thiophenyl;
or a pharmaceutically acceptable salt, stereoisomer or tautomer thereof.

2. A compound according to claim 1, wherein R¹ is alkylisoxazolyl.

3. A compound according to claim 1 or 2, wherein R¹ is methylisoxazolyl.

4. A compound according to any one of claims 1 to 3, wherein R² is methyl-dioxido-7,8-dihydro-5H-thiopyrano[4,3-d]pyrimidinyl, methoxy-5,6,7,8-tetrahydroquinazolinyl, phenyl-5,6,7,8-tetrahydroquinazolinyl, (methoxyphenyl)-1-oxo-1,2-dihydroisoquinolinyl, dimethylphenylaminocarbonylpyridyl, methoxypyrazinyl, pyridyl, naphtalenyl, methoxypyridyl, methoxypyridazinyl, oxo-pyridyl, (N-methyl)oxo-pyridyl, oxo-indanyl, trifluoromethylphenyl, methyloxycarbonylphenyl, (ethoxycarbonyl)methylthiophenyl or (methoxycarbonyl)thiophenyl.

5. A compound according to any one of claims 1 to 4, wherein R² is oxo-pyridyl, (N-methyl)oxo-pyridyl or oxoindanyl.

6. A compound according to any one of claims 1 to 5 selected from
(Z)-2-cyano-3-hydroxy-N-(4-methyl-6,6-dioxido-7,8-dihydro-5H-thiopyrano[4,3-d]pyrimidin-2-yl)-3-(5-methylisoxazol-4-yl)acrylamide;
(Z)-2-cyano-3-hydroxy-N-(4-methoxy-5,6,7,8 -tetrahydroquinazolin-2-yl)-3-(5-methylisoxazol-4-yl)acrylamide;
(Z)-2-cyano-3-hydroxy-3-(5-methylisoxazol-4-yl)-N-(4-phenyl-5,6,7,8-tetrahydroquinazolin-2-yl)acrylamide;
(Z)-2-cyano-3-hydroxy-N-(3-(3-methoxyphenyl)-1-oxo-1,2-dihydroisoquinolin-6-yl)-3-(5-methylisoxazol-4-yl)acrylamide;
(Z)-5-(2-cyano-3-hydroxy-3-(5-methylisoxazol-4-yl)acrylamido)-N-(2,6-dimethylphenyl)picolinamide;
(Z)-2-cyano-3-hydroxy-N-(5-methoxypyrazin-2-yl)-3-(5-methylisoxazol-4-yl)prop-2-enamide;
(Z)-2-cyano-3-hydroxy-3-(5-methylisoxazol-4-yl)-N-(4-pyridyl)prop-2-enamide;
(Z)-2-cyano-3-hydroxy-3-(5-methylisoxazol-4-yl)-N-(naphthalen-2-yl)acrylamide;
(Z)-2-cyano-3-hydroxy-3-(5-methylisoxazol-4-yl)-N-(naphthalen-1-yl)acrylamide;
(Z)-2-cyano-3-hydroxy-N-(6-methoxy-3-pyridyl)-3-(5-methylisoxazol-4-yl)prop-2-enamide;
(Z)-2-cyano-3-hydroxy-N-(6-methoxypyridazin-3-yl)-3-(5-methylisoxazol-4-yl)prop-2-enamide;
(Z)-2-cyano-3-hydroxy-3-(5-methylisoxazol-4-yl)-N-(2-oxo-1H-pyridin-4-yl)prop-2-enamide;
(Z)-2-cyano-3-hydroxy-3-(5-methylisoxazol -4-yl)-N-(1-methyl-2-oxo-4-pyridyl)prop-2-enamide;
(Z)-2-cyano-3-hydroxy-3-(1-methyl-1H-1,2,3-triazol-5-yl)-N-(4-(trifluoromethyl)phenyl)acrylamide;
(Z)-2-cyano-3-hydroxy-3-(5-methylisoxazol-4-yl)-N-(1-oxoindan-5-yl)prop-2-enamide;
(Z)-2-cyano-3-hydroxy-3-(1-methyl-1H-imidazol-5-yl)-N-(4-(trifluoromethyl)phenyl)acrylamide;
(Z)-2-cyano-3-hydroxy-3-(5-methylisoxazol-4-yl)-N-(3-pyridyl)prop-2-enamide;
(Z)-2-cyano-3-hydroxy-3-(5-methylisothiazol-4-yl)-N-(4-(trifluoromethyl)phenyl)acrylamide;
(Z)-2-cyano-3-hydroxy-3-(4-methylisoxazol-5-yl)-N-(4-(trifluoromethyl)phenyl)acrylamide;
(Z)-2-cyano-3-hydroxy-3-(oxazol-5 -yl)-N-(4-(trifluoromethyl)phenyl)acrylamide;
methyl (Z)-4-(2-cyano-3-hydroxy-3-(3-methylisoxazol-4-yl)acrylamido)benzoate;
(Z)-2-cyano-3-hydroxy-3-(3-methylisoxazol-4-yl)-N-(4-(trifluoromethyl)phenyl)acrylamide;
ethyl (Z)-2-(2-cyano-3-hydroxy-3-(5-methylisoxazol-4-yl)acrylamido)-4-methylthiophene-3-carboxylate; and
methyl (Z)-3-(2-cyano-3-hydroxy-3-(5-methylisoxazol-4-yl)acrylamido)thiophene-2-carboxylate;
or a pharmaceutically acceptable salt, stereoisomer or tautomer thereof.

7. A compound according to any one of claims 1 to 6 selected from
(Z)-2-cyano-3-hydroxy-3-(5-methylisoxazol-4-yl)-N-(2-oxo-1H-pyridin-4-yl)prop-2-enamide;
(Z)-2-cyano-3-hydroxy-3-(5-methylisoxazol-4-yl)-N-(1-methyl-2-oxo-4-pyridyl)prop-2-enamide; and
(Z)-2-cyano-3-hydroxy-3-(5-methylisoxazol-4-yl)-N-(1-oxoindan-5-yl)prop-2-enamide;
or a pharmaceutically acceptable salt, stereoisomer or tautomer thereof.

8. A process for the preparation of a compound according to any one of claims 1 to 7, comprising the coupling of a compound of formula (A1) 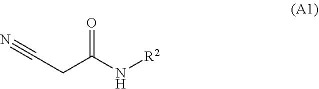
with a compound of formula (A2) 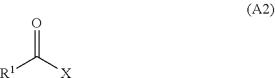
in the presence of a base;
wherein R¹ and R² are as defined in any one of claims 1 to 7 and X is a leaving group, such as a halogen, mesylate or tosylate.

9. A compound according to any one of claims 1 to 7, when manufactured according to a process of claim 8.

10. A compound according to any one of claims 1 to 7, for use as therapeutically active substance.

11. A pharmaceutical composition comprising a compound in accordance with any one of claims 1 to 7 and a therapeutically inert carrier.

12. The use of a compound according to any one of claims 1 to 7 for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

13. The use of a compound according to any one of claims 1 to 7 for the preparation of a medicament for the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

14. A compound according to any one of claims 1 to 7 for use in the treatment or prophylaxis of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS).

15. A method for the treatment of systemic lupus erythrematosus (SLE), cutaneous skin diseases like dermatomyositis or cutaneous lupus, interstitial pulmonary fibrosis, Sjogren syndrome, type I diabetes, inflammatory bowel disease, non-alcoholic steatohepatitis (NASH), juvenile inflammatory arthritis, ankylosing spondylitis, gout or Aicardi-Goutieres syndrome (AGS), which method comprises administering an effective amount of a compound as defined in any one of claims 1 to 7 to a patient in need thereof.

16. The invention as hereinbefore described. ***
